(19) United States
(12) Patent Application Publication
(10) Pub. No.: US 2005/0196464 A1
(43) Pub. Date: Sep. 8, 2005

(54) Method and pharmaceutical composition for treatment of skin neoplasm

(75) Inventors: Yu-Fang Hu, Chungli City (TW); Chi-Liang Kan, Chungho City (TW)

Correspondence Address: BINGHAM, MCCUTCHEN LLP, THREE EMBARCADERO CENTER, 18 FLOOR, SAN FRANCISCO, CA, 94111-4067, US

(73) Assignee: TTY BIOPHARM COMPANY LIMITED, Taipei (TW)

(21) Appl. No.: 10/791,572

(22) Filed: Mar. 3, 2004

(51) Int. Cl. A61K 33/36

(52) U.S. Cl. 424/623

(57) ABSTRACT

The invention relates to a pharmaceutical composition for the treatment of skin neoplasm, which comprises a therapeutically effective amount of arsenic trioxide and a pharmaceutically acceptable carrier. Furthermore, the invention also relates to a method of treating skin neoplasm in a human by administering to a human in need thereof a therapeutically effective amount of a pharmaceutical composition comprising arsenic trioxide.

### BACKGROUND OF THE INVENTION

1. Field of the Invention

The present invention relates to a method and pharmaceutical composition for the treatment of skin neoplasm, and more particularly to the novel uses of arsenic trioxide for treating skin neoplasm.

2. Description of Related Art

Arsenic trioxide has been considered to be both a poison and a drug for a long time in both Western and Chinese medical practices. Because of the known carcinogenic effect of arsenic trioxide, arsenic trioxide was only used in Western medicine to treat tropical diseases such as African trypanosomiasis. In the latter part of the nineteenth century, arsenic trioxide was used frequently in attempts to treat diseases of the blood in the West. In traditional Chinese medicine, arsenic trioxide has been used to treat tooth marrow diseases, psoriasis, syphilis and rheumatosis.

In recent years, arsenic trioxide was reported as a treatment for leukemic patients with markedly reduced white blood cell count. Further reports described that arsenic trioxide can treat acute promyelocytic leukemia (Blood, 88(3): 1052-1061, 1996; European Journal of Cancer, 35(8): 1258-1263, 1999; and The New England Journal of Medicine, 339(19): 1341-1348, 1998).

Although arsenic trioxide is well known to be both a poison and a carcinogenic agent, many people are studying the use of arsenic trioxide in medical treatment.

### SUMMARY OF THE INVENTION

An aspect of the present invention is to provide a pharmaceutical composition for the treatment of skin neoplasm. The pharmaceutical composition comprises a therapeutically effective amount of arsenic trioxide and a pharmaceutically acceptable carrier.

Another aspect of the present invention is to provide a method for treating skin neoplasm in a human. The method comprises administering to a human in need of treatment for skin neoplasm a therapeutically effective amount of a pharmaceutical composition comprising arsenic trioxide.

Further benefits and advantages of the present invention will become apparent after a careful reading of the detailed description with appropriate reference to the accompanying drawings.

### BRIEF DESCRIPTION OF THE DRAWINGS

FIG. 1 is a graph showing anti-tumor therapeutic efficacy of arsenic trioxide administered to the wounded skin of mice with time; and

FIG. 2 is a graph showing anti-tumor therapeutic efficacy of arsenic trioxide administered to the skin of mice with time.

### DETAILED DESCRIPTION OF THE INVENTION

The term “therapeutically effective amount” as used herein means a nontoxic but sufficient amount of an active agent to provide the desired therapeutic effect.

The term “administered topically” as used herein is used in its conventional sense to mean delivery of a topical drug of a pharmacologically active agent to the skin, as in, for example, the treatment of various skin neoplasm. Topical drug administration provides a local rather than a systemic effect.

The term “carrier” as used herein refers to carrier material suitable for topical drug administration. Carriers useful herein include any such materials known in the art, which are nontoxic and do not interact with other components of the composition in a deleterious manner.

Arsenic trioxide is white or transparent non-crystal lumps or crystal, difficult to dissolve in water, having a sweet taste and is poison. Arsenic trioxide is usually used in manufacturing glass or enamel materials.

In a preferred embodiment of the present invention, the treatment of subcutaneous tumors in animal experiments with arsenic trioxide obviously suppressed the tumor growth whether the tumor was incised or not. Arsenic trioxide employed according to the present invention can be used to treat skin neoplasm. Preferably, arsenic trioxide employed according to the present invention can be used for treatment of subcutaneous tumors, primary skin cancer, melanomatous cancer or metastatic cutaneous cancer. The primary skin cancer includes basic cell carcinoma, squamous cell carcinoma and Merkel cell carcinoma.

The present invention provides a pharmaceutical composition, a dosage form and a method for the treatment of skin neoplasm. The pharmaceutical composition for the treatment of skin neoplasm in accordance with the present invention comprises a therapeutically effective amount of arsenic trioxide and a pharmaceutically acceptable carrier. The method for treating of skin neoplasm in accordance with the present invention comprises administering to a human in need thereof a therapeutically effective amount of a pharmaceutical composition comprising arsenic trioxide.

In a preferred embodiment of the present invention, a dosage form suitable for topical administration which comprises the foregoing composition. Preferably, the dosage form comprises viscid substance for preventing arsenic trioxide from spreading in the air. The viscid dosage form can prevent patient or medical personnel from inhaling arsenic trioxide. Preferably, the viscid substance is starch or polymer. More preferably, the polymer is polyarylic acid, carbomer, hydroxyethyl cellulose, chitosan, hydroxypropyl cellulose, hydroxypropyl methylcellulose, sodium carboxymethylcellulose, polyvinyl alcohol or a mixture thereof.

In a preferred embodiment of the present invention, the dosage form is in the form of a gel. Preferably, the gel comprises by weight based on the total weight of the gel
0.05% arsenic trioxide;
1.5% carbomer 940;
5.5% PEG; and
0.125% propry paraben.

In an embodiment of the present invention, arsenic trioxide can be used as the active ingredient combination with a pharmaceutical carrier according to conventional pharmaceutical techniques. The carrier may include a wide variety of forms depending on the form of preparation desired for administration, e.g., topical administration. In preparing the compositions for topical administration dosage form, any of the usual pharmaceutical media may be employed, e.g., water, glycols, oils, alcohols, flavoring agents, preservatives, coloring agents, and the like.

The therapeutically effective amount of arsenic trioxide for specific skin neoplasm will vary with the severity of the condition to be treated and the route of administration. The therapeutically effective amount of arsenic trioxide will also vary according to the age, body weight, condition and response of the individual patient. In general, the therapeutically effective amount of arsenic trioxide for the conditions described herein are generally from 0.01 to 1 mg/g of the composition administered topically. A preferred therapeutically effective amount of arsenic trioxide is from 0.1 to 0.5 mg/g of the composition.

The composition may be in any form suitable for application to the body surface and may comprise, for example, a cream, lotion, solution, suspension, gel, ointment, paste, balm, spray, emulsion or the like. Preferably, the composition is in the form of a cream, a lotion or a gel. The composition may be directly applied to the body surface. The composition is administered preferably topically. Preferably, the composition may be contained in a patch or bandaging materials.

All of the documents or publications recited in the text are incorporated herein by reference.

Further details of this invention are illustrated in the following examples.

### EXAMPLE 1

Preparation of a Lotion of Arsenic Trioxide

| | | |
|---|---|---|
| Compound | Amount | Composition (%) |

| | | | |
|---|---|---|---|
| Arsenic trioxide | 0.33 | mg | 0.033 |
| Stearic acid | 9.6 | mg | 0.96 |
| Cetyl alcohol | 7.2 | mg | 0.72 |
| Anhydrous Lanolin | 80 | mg | 8.0 |
| Vegetable oil | 12.8 | mg | 1.28 |
| Triethanolamine | 16 | mg | 1.6 |
| Water | 873.65 | mg | 87.4 |
| Total | 1 | g | 100 |

### EXAMPLE 2

Preparation of a gel of arsenic trioxide

| | | |
|---|---|---|
| Compound | Amount | Composition (%) |

| | | | |
|---|---|---|---|
| Arsenic trioxide | 2 | g | 0.05 |
| Carbomer 940 | 60 | g | 1.5 |
| PEG | 220 | g | 5.5 |
| Propry paraben | 5 | g | 0.125 |
| Water | 3713 | g | 92.825 |
| Total | 4000 | g | 100 |

### EXAMPLE 3

### Effect of Arsenic Trioxide on Subcutaneous Tumors by Topical Administration

HTB-9 cell line (bladder cancer cell line, purchased from American Type Culture Collection, No. ATCC HTB-9) was cultured in a plate with RPMI (10% FCS) in an incubator at 37° C./5% CO₂. When the cells were grown to a sufficient amount, the cells were treated with trypsin to separate them from the plate, then washed with PBS and diluted to a suitable concentration (2×10⁶ cell/50 μl). The diluted cells were inoculated subcutaneously near the forelimb or hind legs of 18 BALB/c-Hfhllnu female mice (8 weeks old), and tumor growth was recorded.

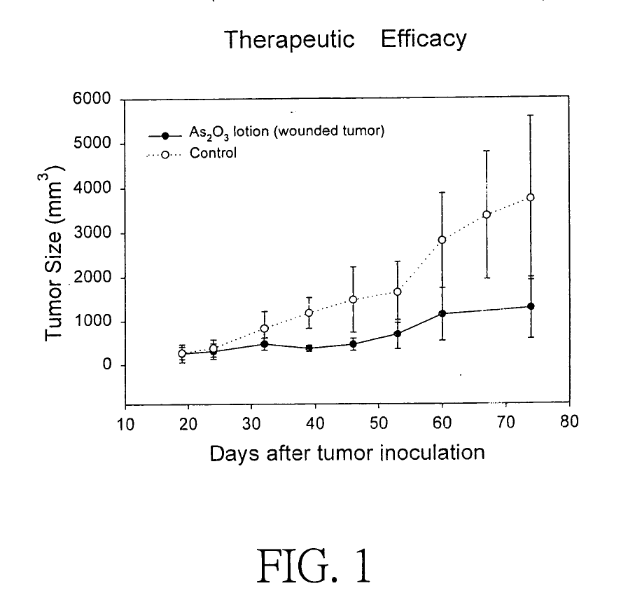

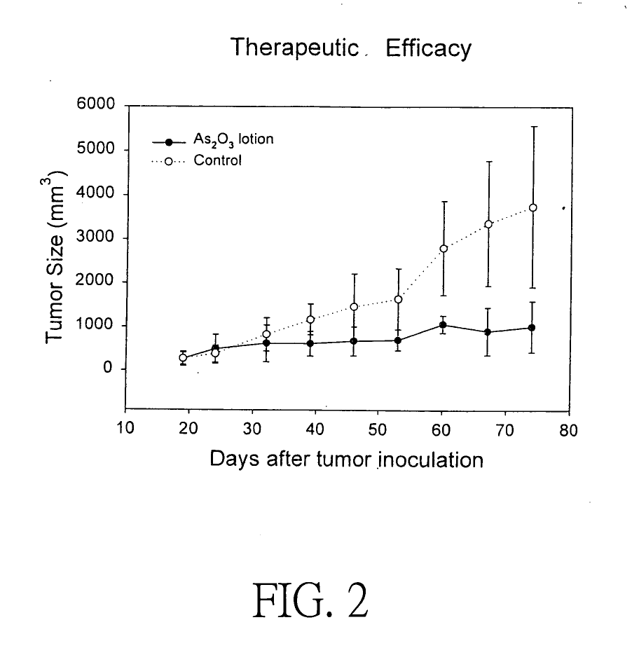

When the tumor was growing after 19 days (the tumor size is about 240 mm³), the tested mice were divided into 3 groups (6 mice/group), and arsenic trioxide started to be administrated. The lotion manufactured in example 1 was administered to the first group at the tumor location of the mice. The second group was the control group, and no drugs were administered. The lotion manufactured in example 1 was administered to the third group to the tumor that was incised. The lotion was administrated 3 times per week (Monday, Wednesday and Friday) and 1 drop each time (about 0.14 g). The health of those three groups including the survival day and the weight of the mice was recorded. The tumor length, the width and the height were measured, and the volume (mm³) [length (mm)×width (mm)×height (mm)] of the tumor was calculated. With reference to FIGS. 1 and 2, treatment of the tumor with the lotion for 80 days obviously suppressed the tumor growth whether the tumor was incised or not.

Although the invention has been explained in relation to its preferred embodiment, many other possible modifications and variations can be made without departing from the spirit and scope of the invention as hereinafter claimed.

1. A pharmaceutical composition for the treatment of skin neoplasm comprising a therapeutically effective amount of arsenic trioxide and a pharmaceutically acceptable carrier.

2. The pharmaceutical composition as claimed in claim 1, wherein the skin neoplasm is selected from the group consisting of subcutaneous tumors, primary skin cancer, melanomatous cancer and metastatic cutaneous cancer.

3. The pharmaceutical composition as claimed in claim 2, wherein the primary skin cancer is selected from the group consisting of basic cell carcinoma, squamous cell carcinoma and Merkel cell carcinoma.

4. The pharmaceutical composition as claimed in claim 3 in the form of a cream, lotion, gel, ointment or paste.

5. A dosage form suitable for topical administration which comprises a composition as claimed in claim 3.

6. The dosage form as claimed in claim 5, wherein the dosage form further comprises viscid substance.

7. The dosage form as claimed in claim 6, wherein the viscid substance is starch or polymer.

8. The dosage form as claimed in claim 7, wherein the polymer is polyarylic acid, carbomer, hydroxyethyl cellulose, chitosan, hydroxypropyl cellulose, hydroxypropyl methylcellulose, sodium carboxymethylcellulose, polyvinyl alcohol or a mixture thereof.

9. The dosage form as claimed in claim 8 in the form of a gel.

10. The dosage form as claimed in claim 9, wherein the gel comprises by weight based on the total weight of the gel
0.05% arsenic trioxide;
1.5% carbomer 940;
5.5% PEG; and
0.125% propry paraben.

11. A method of treating skin neoplasm in a human comprising administering to a human in need thereof a therapeutically effective amount of a pharmaceutical composition comprising arsenic trioxide.

12. A method as claimed in claim 11, wherein the skin neoplasm is selected from the group consisting of subcutaneous tumors, primary skin cancer, melanomatous cancer and metastatic cutaneous cancer.

13. A method as claimed in claim 12, wherein the primary skin cancer is selected from the group consisting of basic cell carcinoma, squamous cell carcinoma and Merkel cell carcinoma.

14. A method as claimed in claim 13, wherein the composition is in the form of a cream, lotion, solution, gel, ointment or paste.

15. A method as claimed in claim 14, wherein the composition is administered topically.

16. A method as claimed in claim 15, wherein arsenic trioxide is present at about 0.01 to about 1 mg/g of the composition.

17. A method as claimed in claim 15, wherein arsenic trioxide is present at about 0.1 to about 0.5 mg/g of the composition.
